(19) United States
(12) Patent Application Publication
(10) Pub. No.: US 2014/0378422 A1
(43) Pub. Date: Dec. 25, 2014

(54) Combination of a RTK inhibitor with an anti-estrogen and use thereof for the treatment of cancer

(71) Applicant: NOVARTIS AG, Basel (CH)

(72) Inventors: Alejandro Yovine, FoLgensbourg (FR); Matthew Squires, Basel (CH); Catherne Reddick, Orange, NJ (US); Yong Zhang, East Hanover, NJ (US)

(73) Assignee: NOVARTIS AG, Basel (CH)

(21) Appl. No.: 14/375,145

(22) PCT Filed: Jan. 30, 2013
(86) PCT No.: PCT/US2013/023781

§ 371 Date: Jul. 29, 2014

Related U.S. Application Data
(60) Provisional application No. 61/593,047, filed on Jan. 31, 2012.

(51) Int. Cl. A61K 31/496; A61K 31/565

(52) U.S. Cl. CPC A61K 31/496; A61K 31/565
USPC 514/171

(57) ABSTRACT

A pharmaceutical combination comprising (a) a RTK inhibitor selected from the group consisting of compounds of Formula I or a tautomer thereof, compounds of Formula II or a tautomer thereof, compounds of Formula III or a tautomer thereof, a pharmaceutically acceptable salt of the compound, a pharmaceutically acceptable salt of the tautomer, or a mixture thereof; and (b) one or more anti-estrogen compounds, or a pharmaceutically acceptable salt thereof; such as tamoxifen, toremifene, fulvestrant, raloxifene or raloxifene hydrochloride; the uses of such combination in the treatment or prevention of proliferative diseases: and methods of treating a subject suffering from a proliferative disease; and methods of treating a subject suffering from a proliferative disease comprising administering a therapeutically effective amount of such combination.

### FIELD OF THE INVENTION

A pharmaceutical combination comprising: (a) at least one receptor tyrosine kinase (RTK) inhibitor compounds targeting/decreasing a protein or lipid kinase activity, selected from the group consisting of compounds of Formula I or a tautomer thereof, compounds of Formula II or a tautomer thereof, compounds of Formula III or a tautomer thereof, a pharmaceutically acceptable salt of the compound, a pharmaceutically acceptable salt of the tautomer, or a mixture thereof; and (b) one or more anti-estrogen compounds, or a pharmaceutically acceptable salt thereof; such as tamoxifen, toremifene, fulvestrant, raloxifene or raloxifene hydrochloride; the uses of such combination in the treatment or prevention of proliferative diseases; and methods of treating a subject suffering from a proliferative disease comprising administering a therapeutically effective amount of such combination.

### BACKGROUND OF THE INVENTION

The compounds of Formula I, Formula II and Formula III inhibit various protein kinases, such as receptor tyrosine kinases (RTKs). Receptor tyrosine kinases (RTKs) are transmembrane polypeptides that regulate developmental cell growth and differentiation, remodeling and regeneration of adult tissues. Polypeptide ligands known as growth factors or cytokines, are known to activate RTKs. Signalling RTKs involves ligand binding and a shift in conformation in the external domain of the receptor resulting in its dimerization. Binding of the ligand to the RTK results in receptor trans-phosphorylation at specific tyrosine residues and subsequent activation of the catalytic domains for the phosphorylation of cytoplasmic substrates.

Two subfamilies of RTKs are specific to the vascular endothelium. These include the vascular endothelial growth factor (VEGF) subfamily and the Tie receptor subfamily. Class III RTKs include vascular endothelial growth factor receptor 1 (VEGFR-1), vascular endothelial growth factor receptor 2 (VEGFR-2), and vascular endothelial growth factor receptor 3 (VEGFR-3).

Inhibited tyrosine kinases include Cdc2 kinase (cell division cycle 2 kinase), Fyn (FYN oncogene kinase related to SRC, FGR, YES), Lck (lymphocyte-specific protein tyrosine kinase), c-Kit (stem cell factor receptor or mast cell growth factor receptor), p60src (tyrosine kinase originally identified as the v-src oncogene of the rous sarcoma virus), c-ABL (tyrosine kinase that stands for an oncogene product originally isolated from the Adelson leukemia virus), VEGFR3, PDGFRα (platelet derived growth factor receptor α), PDGFRβ (platelet derived growth factor receptor β), FGFR3 (fibroblast growth factor receptor 3), FLT-3 (fins-like tyrosine kinase-3), or Tie-2 (tyrosine kinase with Ig and EGF homology domains).

In spite of numerous treatment options for proliferative disease patients and cancer patients, there remains a need for effective and safe antiproliferative agents and a need for new combination therapies that can be administered for the effective long-term treatment of proliferative diseases such as cancer.

### SUMMARY OF THE INVENTION

The present invention relates to a pharmaceutical combination comprising: (a) at least one RTK inhibitor compound, selected from the group consisting of compounds of Formula I or a tautomer thereof, compounds of Formula II or a tautomer thereof, compounds of Formula III or a tautomer thereof, a pharmaceutically acceptable salt of the compound, a pharmaceutically acceptable salt of the tautomer, or a mixture thereof; and (b) one or more anti-estrogen compounds, or a pharmaceutically acceptable salt thereof; such as tamoxifen, toremifene, fulvestrant, raloxifene or raloxifene hydrochloride; for simultaneous, separate or sequential administration, in particular for treating or preventing a proliferative disease.

The present invention also pertains to a combination such as a combined preparation of a pharmaceutical combination comprising: (a) at least one RTK inhibitor compound selected from the group consisting of compounds of Formula I or a tautomer thereof, compounds of Formula II or a tautomer thereof, compounds of Formula III or a tautomer thereof, a pharmaceutically acceptable salt of the compound, a pharmaceutically acceptable sale of the tautomer, or a mixture thereof; and (b) one or more anti-estrogen compounds, or a pharmaceutically acceptable salt thereof; such as tamoxifen, toremifene, fulvestrant, raloxifene or raloxifene hydrochloride.

The present invention particularly pertains to a COMBINATION OF THE INVENTION useful for treating or preventing a proliferative disease in a subject in need thereof.

The present invention also pertains to a COMBINATION OF THE INVENTION for use in the preparation of a pharmaceutical composition or medicament for the treatment or prevention of a proliferative disease in a subject in need thereof.

The present invention further pertains to the use of a RTK inhibitor compound selected from the group consisting of compounds of Formula I or a tautomer thereof, compounds of Formula II or a tautomer thereof, compounds of Formula III or a tautomer thereof, or a pharmaceutically acceptable salt of the compound, a pharmaceutically acceptable salt of the tautomer, or a mixture thereof; in combination with at least one anti-estrogen compound, or a pharmaceutically acceptable salt thereof; such as tamoxifen, toremifene, fulvestrant, raloxifene or raloxifene hydrochloride; for the preparation of a pharmaceutical composition or medicament for the treatment of prevention of a proliferative disease.

The present invention relates to a method of treating a subject having a proliferative disease comprising administered to said subject a COMBINATION OF THE INVENTION in a quantity, which is jointly therapeutically effective against a proliferative disease.

The present invention further provides a commercial package comprising as therapeutic agents a COMBINATION OF THE INVENTION, together with instructions for simultaneous, separate or sequential administration thereof for use in the delay of progression or treatment of a proliferative disease.

### BRIEF DESCRIPTION OF THE FIGURES

FIG. 1

The effect of combining pan-FGFR inhibitor Compound B with ER antagonist Fulvestrant in either standard (A) or steroid hormone depleted (B) media. Shown in the upper two grids in A and B (labeled percent growth inhibition) are the growth inhibition values for each single agent and combination treatment relative to DMSO with numerical values representing the percentage of growth inhibition relative to control cells. In each grid the effect on the growth of cells of increasing concentrations of Compound B are shown along the bottom rows from left to right and increasing concentrations of Fulvestrant along the leftmost columns from bottom to top. All remaining points in the grids display the percent inhibition of growth that results from a combination of the two inhibitors that corresponds to the single agent concentrations denoted on the two axes. Displayed in the bottom grids in A and B (labeled excess inhibition) is a measure of the excess inhibition observed for each corresponding point in the upper grid. Excess inhibition was calculated using the Lowe synergy model which measures the effect on growth relative to what would be expected if two drugs behave in a dose-additive manner.

FIG. 2

A. Compound B has a modest effect on proliferation as a single agent concentrations≧1.3 uM. Fulvestrant has a modest effect on proliferation as a single agent at concentrations≧0.049 uM. Combining Compound B and Fulvestrant results in synergy at Compound B concentrations≧1.3 uM (synergy score=0.7).

B. No combination effects were observed between the compounds in steroid-depleted media (synergy score=0).

### DETAILED DESCRIPTION OF THE INVENTION

The present invention relates to a pharmaceutical combination comprising: (a) at least one RTK inhibitor compound selected from the group consisting of compounds of Formula I or a tautomer thereof, compounds of Formula II or a tautomer thereof, compounds of Formula III or a tautomer thereof, a pharmaceutically acceptable salt of the compound, a pharmaceutically acceptable salt of the tautomer, or a mixture thereof; and (b) one or more anti-estrogen compounds, or a pharmaceutically acceptable salt thereof; such as tamoxifen, toremifene, fulvestrant, raloxifene or raloxifene hydrochloride; for simultaneous, separate or sequential administration, in particular for treating or preventing a proliferative disease.

Preliminary results from a clinical trial using COMPOUND A in metastatic breast cancer patient suggest that Compound A may be more efficacious in patients with FGF amplified disease. In this trial, Compound A was used as a single agent in a heavily pre-treated population with extensive visceral involvement, including liver metastases in most cases. Applicants discovered that the data suggested a potential signal in this population of patients with poor prognosis, particularly in the subset of patients with FGF amplified disease (FGFR1 and/or FGFR2 and/or FGF3 amplification) where 3 unconfirmed partial responses and five patients with stable disease for greater than 24 weeks and/or a partial response that was not confirmed at four weeks, was observed. In addition, patients with FGF-pathway non-amplified disease also showed long-lasting disease stabilization, where 3 patients had stable disease greater than 24 weeks. Although proof-of-concept (PoC) was not achieved in this trial, these data were regarded as encouraging in this poor-prognosis population of metastatic breast cancer patients. Applicants thus discovered that this subpopulation of patients likely would respond much better with the combination of Compound A and fulvestrant, rather than fulvestrant alone. Compound A in combination with fulvestrant in a less pretreated population will provide even more improved outcomes for the treated patients.

Biomarker exploratory data also revealed that FGF23, as surrogate marker of FGFR 1 inhibition, was increased above baseline during COMPOUND A treatment, thereby confirming that Compound A inhibited FGFR1. Based on this information, the amplified as well as non-amplified group of patients are included in the COMPOUND A trial for test kit validation and for an assessment of any off FGF-pathway target activity.

In accordance with the present invention, outcomes in patients with HR+/HER2− locally advanced or metastatic breast cancer are improved when COMPOUND A is combined with fulvestrant.

In respect to potential drug-drug interactions (DDIs) and potential toxicities as a result of the combination therapy, applicants have discovered that fulvestrant in combination with Compound A does not cause significant CYP3A4-mediated drug interactions. Applicants have also discovered that fulvestrant plus Compound A combination therapy is favorable in HR+ and HER2− breast cancer patients as this combination has the limited overlapping toxicities for both drugs (e.g., nausea, fatigue and liver function test abnormalities, etc.).

The combination of an effective amount of the tyrosine kinase inhibitor compounds of Formula I, Formula II and Formula III, such as Compound A, with an effective amount of an anti-estrogen compound, such as fulvestrant, results in significant improvement in the treatment of proliferative diseases, such as breast cancer, and in particular, HR+/HER2− breast cancer, preferably where patients have evidence of disease progression on prior endocrine therapy.

When administered simultaneously, sequentially or separately, the tyrosine kinase inhibitor compounds of Formula I, such as Compound A, and an anti-estrogen compound, such as fulvestrant, interact in a synergistic manner to inhibit cell proliferation.

Estrogen deprivation is fundamental to the treatment of many benign and malignant diseases of the breast and reproductive tract. In premenopausal women, this is achieved by the ablation of ovarian function through surgical, radiotherapeutic, or medical means, and, in postmenopausal women, by the use of aromatase inhibitors. An alternative approach to oestrogen withdrawal is to antagonise oestrogens with anti-estrogens. These are drugs that bind to and compete for estrogen receptors (ER) present in the nuclei of oestrogen-responsive tissue.

Overcoming de novo or acquired endocrine resistance remains critical to enhancing the benefit of available compounds in patients with breast cancer, and in particular hormone receptor positive (HR+) breast cancer. Recent progress has been made in understanding the molecular biology associated with acquired endocrine resistance, including adaptive “cross-talk” between ER and peptide growth factor receptor pathways, such as the fibroblast growth factor receptor (FGFR). Up to 8% of HR+/human epidermal growth factor receptor 2 negative (HER2−) breast cancer patients have amplification of the FGFR1 gene, which is associated with resistance to endocrine therapy, but can be overcome via FGFR1 inhibition in preclinical models.

The compounds of Formula I, Formula II and Formula III are potent VEGF, PDGF, and FGF receptor tyrosine kinase inhibitors that demonstrated antitumor activity in heavily pretreated breast cancer pts with FGF pathway amplification (FGFR1 and/or FGFR2 and/or ligand FGF3). The compounds of Formula I, Formula II and Formula III reverse resistance to endocrine therapy related to FGF-pathway amplification and, improve outcomes when combined with fulvestrant.

Fulvestrant is currently approved for HR+ postmenopausal breast cancer patients after recurrence or progression on anti-estrogen therapy; however there is considerable evidence showing that fulvestrant is effective not only after failure on tamoxifen, but also after failure on aromatase inhibitors, from 1^{st} to 3^{rd} line settings. Indeed, several treatment guidelines, e.g., the NCCN treatment Guidelines, include fulvestrant as a treatment option for HR+/HER2− patients with bone or soft tissue only or asymptomatic visceral disease.

By competitively binding to the estrogen receptor, fulvestrant effectively down regulates the receptor, leading to its rapid degradation. In preclinical models fulvestrant dramatically reverses endocrine resistance associated with ligand-independent ER activation, which is at least in part responsible for resistance to aromatase inhibitors and tamoxifen. On the other hand, Compound A, which inhibits FGFR1, may reverse resistance to endocrine therapy related in part as well to FGF pathway amplification, and thus improve outcomes when combined with fulvestrant. Moreover, Compound A also has anti-angiogenic effect as it targets VEGF/PDGFR; and since angiogenesis plays an essential role in breast cancer development, efficacy in breast cancer treatment is improved with the fulvestrant plus Compound A combination therapy.

In addition, there are limited overlapping toxicities for both drugs (e.g., nausea, fatigue and liver function test abnormalities, etc.) suggesting that fulvestrant+Compound A combination therapy may be favorable in HER2− and HR+ breast cancer patients that have evidence of disease progression on prior endocrine therapy.

The general terms used herein are defined with the following meanings, unless explicitly stated otherwise:

The terms “comprising” and “including” are used herein in their open-ended and nonlimiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The term “combination” or “pharmaceutical combination”, as used herein, defines either a fixed combination in one dosage unit form, or non-fixed combination (or a kit of parts) for the combined administration where a compound of the Formula I, Formula II or Formula III and a combination partner (e.g. an anti-estrogen drug as explained below, also referred to as “therapeutic agent” or “co-agent”) may be administered independently at the same time or separately within time intervals, especially where these time intervals allow that the combination partners show a cooperative, e.g. synergistic effect. The term “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “fixed combination” means that the active ingredients, e.g. a compound of Formula (I), Formula II or Formula III and a combination partner, are both administered to a patient simultaneously in the form of a single entity or dosage. The terms “non-fixed combination” or “kit of parts” mean that the active ingredients, e.g. a compound of Formula I, Formula II or Formula III and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The term “anti-estrogen”, as used herein, relates to a compound which antagonizes the effect of estrogens at the estrogen receptor level. The term includes, but is not limited to, tamoxifen, toremifene, fulvestrant, raloxifene and raloxifene hydrochloride. Tamoxifen can be administered in the form as it is marketed, e.g., NOLVADEX; and raloxifene hydrochloride is marketed as EVISTA. Toremifene can by administered in the form as it is marketed, e.g., FARESTON. Fulvestrant can be formulated as disclosed in U.S. Pat. No. 4,659,516 and is marketed as FASLODEX. A combination of the invention comprising a pharmaceutically active agent which is an anti-estrogen is particularly useful for the treatment of estrogen receptor positive tumors, e.g., breast tumors.

The term “RTK inhibitors” as used herein, includes, but is not limited to, protein tyrosine kinase and/or serine and/or threonine kinase inhibitors or lipid kinase inhibitors, for example:
i) compounds targeting, decreasing or inhibiting the activity of the vascular endothelial growth factor-receptors (VEGF), such as compounds which target, decrease or inhibit the activity of VEGF, especially compounds which inhibit the VEGF receptor, such as, but not limited to, 7H-pyrrolo[2,3-d]pyrimidine derivatives (AEE788); BAY 43-9006: isocholine compounds disclosed in WO 00/09495 such as (4-tert-butyl-phenyl)-94-pyridin-4-ylmethyl-isoquinolin-1-yl)-amine (AAL881); and
ii) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g., a N-phenyl-2-pyrimidine-amine derivative, e.g., imatinib, SU101, SU6668 and GFB-111;
iii) compounds targeting, decreasing or inhibiting the activity of the fibroblast growth factor-receptors (FGFR);
iv) compounds targeting, decreasing or inhibiting the activity of the Trk receptor tyrosine kinase family;
v) compounds targeting, decreasing or inhibiting the activity of the FLT3 receptor tyrosine kinase family; and
vi) compounds targeting, decreasing or inhibiting the activity of the C-kit receptor tyrosine kinases (part of the PDGFR family), such as compounds which target, decrease or inhibit the activity of the c-Kit receptor tyrosine kinase family, especially compounds which inhibit the c-Kit receptor, e.g., imatinib; and
vii) compounds targeting, decreasing or inhibiting the activity of protein-tyrosine kinase, such as imatinib mesylate (GLEEVEC); tyrphostin or pyrymidylaminobenzamide and derivatives thereof (AMN 107). A tyrphostin is preferably a low molecular weight (Mᵣ<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810, AG 99, Tyrphostin AG 213, Tyrphostin AG 1748, Tyrphostin AG 490, Tyrphostin B44, Tyrphostin B44 (+) enantiomer, Tyrphostin AG 555, AG 494, Tyrphostin AG 556; AG957 and adaphostin (4-{[(2,5-dihydroxyphenyl)methyl]amino}-benzoic acid adamantyl ester, NSC 680410, adaphostin).

The term “pharmaceutical composition” is defined herein to refer to a mixture or solution containing at least one therapeutic agent to be administered to a subject, e.g., a mammal or human, in order to prevent or treat a particular disease or condition affecting the mammal.

The term “pharmaceutically acceptable” is defined herein to refer to those compounds, materials, compositions and for dosage forms, which are, within the scope of sound medical judgment, suitable for contact with the tissues a subject, e.g., a mammal or human, without excessive toxicity, irritation allergic response and other problem complications commensurate with a reasonable benefit/risk ratio.

The term “co-administration” or “combined administration” as used herein is defined to encompass the administration of the selected therapeutic agents to a single patient, and are intended to include treatment regimens in which the agents are not necessarily administered by the same route of administration or at the same time.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present invention, the term “treat” also denotes to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject.

The term “prevent”, “preventing” or “prevention” as used herein comprises the prevention of at least one symptom associated with or caused by the state, disease or disorder being prevented.

The term “jointly therapeutically active” or “joint therapeutic effect” means that the therapeutic agents may be given separately (in a chronologically staggered manner, especially a sequence-specific manner) in such time intervals that they prefer, in the warm-blooded animal, especially human, to be treated, still show a (preferably synergistic) interaction (joint therapeutic effect). Whether this is the case can, inter alia, be determined by following the blood levels, showing that both compounds are present in the blood of the human to be treated at least during certain time intervals.

The term “pharmaceutically effective amount” or “clinically effective amount” of a combination of therapeutic agents is an amount sufficient to provide an observable improvement over the baseline clinically observable signs and symptoms of the disorder treated with the combination.

The term “subject” or “patient” as used herein includes animals, which are capable of suffering from or afflicted with a cancer or any disorder involving, directly or indirectly, a cancer. Examples of subjects include mammals, e.g., humans, dogs, cows, horses, pigs, sheep, goats, cats, mice, rabbits rats and transgenic non-human animals. In the preferred embodiment, the subject is a human, e.g., a human suffering from, at risk of suffering from, or potentially capable of suffering from cancers.

The term about” or “approximately” shall have the meaning of within 10%, more preferably within 5%, of a given value or range.

Generally, reference to a certain element such as hydrogen or H is meant to include all isotopes of that element. For example, if an R group is defined to include hydrogen or H, it also includes deuterium and tritium.

The phrase “unsubstituted alkyl” refers to alkyl groups that do not contain heteroatoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, —CH(CH₂CH₃)CH(CH₃)CH(CH₃)(CH₂CH₃), and others. The phrase also includes cyclic alkyl groups such as cycloalkyl groups such as cyclopropyl, cyclobutyl, cyclopentyl, cyclohexyl, cycloheptyl, and cyclooctyl and such rings substituted with straight and branched chain alkyl groups as defined above. The phrase also includes polycyclic alkyl groups such as, but not limited to, adamantyl norbornyl, and bicyclo[2.2.2]octyl and such rings substituted with straight and branched chain alkyl groups as defined above. Thus, the phrase unsubstituted alkyl groups includes primary alkyl groups, secondary alkyl groups, and tertiary alkyl groups. Unsubstituted alkyl groups may be bonded to one or more carbon atom(s), oxygen atom(s), nitrogen atom(s), and/or sulfur atom(s) in the parent compound. Preferred unsubstituted alkyl groups include straight and branched chain alkyl groups and cyclic alkyl groups having 1 to 20 carbon atoms. More preferred such unsubstituted alkyl groups have from 1 to 10 carbon atoms while even more preferred such groups have from 1 to 5 carbon atoms. Most preferred unsubstituted alkyl groups include straight and branched chain alkyl groups having from 1 to 4 or from 1 to 3 carbon atoms and include methyl, ethyl, propyl, and —CH(CH₃)₂.

The phrase “substituted alkyl” refers to an unsubstituted alkyl group as defined above in which one or more bonds to a carbon(s) or hydrogen(s) are replaced by a bond to non-hydrogen and non-carbon atoms such as, but not limited to, a halogen atom in halides such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups; and other heteroatoms in various other groups. Substituted alkyl groups also include groups in which one or more bonds to a carbon(s) or hydrogen(s) atom is replaced by a bond to a heteroatom such as oxygen in carbonyl, carboxyl, and ester groups; nitrogen in groups such as imines, oximes, hydrazones, and nitriles. Preferred substituted alkyl groups include, among others, alkyl groups in which one or more bonds to a carbon or hydrogen atom is/are replaced by one or more bonds to fluorine atoms. One example of a substituted alkyl group is the trifluoromethyl group and other alkyl groups that contain the trifluoromethyl group. Other alkyl groups include those in which one or more bonds to a carbon or hydrogen atom is replaced by a bond to an oxygen atom such that the substituted alkyl group contains a hydroxyl, alkoxy, aryloxy group, or heterocyclyloxy group. Still other alkyl groups include alkyl groups that have an amine, alkylamine, dialkylamine, arylamine, (alkyl)(aryl)amine, diarylamine, heterocyclylamine, (alkyl)(heterocyclyl)amine, (arylXheterocyclyl)amine, or diheterocyclylamine group.

The phrase “unsubstituted aryl” refers to aryl groups that do not contain heteroatoms. Thus, by way of example, the phrase includes, but is not limited to, groups such as phenyl, biphenyl, anthracenyl, and naphthyl. Although the phrase “unsubstituted aryl” includes groups containing condensed rings such as naphthalene, it does not include aryl groups that have other groups such as alkyl or halo groups bonded to one of the ring members, as aryl groups such as tolyl are considered herein to be substituted aryl groups as described below. A preferred unsubstituted aryl group is phenyl. In some embodiments, unsubstituted aryl groups have from 6 to 14 carbon atoms. Unsubstituted aryl groups may be bonded to one or more carbon atom(s), oxygen atom(s), nitrogen atom(s), and/or sulfur atom(s) in the parent compound.

The phrase “substituted aryl group” has the same meaning with respect to unsubstituted aryl groups that substituted alkyl groups had with respect to unsubstituted alkyl groups. However, a substituted aryl group also includes aryl groups in which one of the aromatic carbons is bonded to one of the non-carbon or non-hydrogen atoms described above and also includes aryl groups in which one or more aromatic carbons of the aryl group is bonded to a substituted or unsubstituted alkyl, alkenyl, or alkynyl group as defined herein. This includes bonding arrangements in which two carbon atoms of an aryl group are bonded to two atoms of an alkyl, alkenyl, or alkynyl group to define a fused ring system (e.g. dihydronaphthyl or tetrahydronaphthyl). Thus, the phrase “substituted aryl” includes, but is not limited to groups such as tolyl, and hydroxyphenyl among others.

The phrase “unsubstituted alkenyl” refers to straight and branched chain and cyclic groups such as those described with respect to unsubstituted alkyl groups as defined above, except that at least one double bond exists between two carbon atoms. Examples include, but are not limited to vinyl, —CH═C(H)(CH₃), —CH═C(CH₃)₂, —C(CH₃)═C(H)₂, —C(CH₃)═C(H)(CH₃), —C(CH₂CH₃)═CH₂, cyclohexenyl, cyclopentenyl, cyclohexadienyl, butadienyl, pentadienyl, and hexadienyl among others. In some embodiments, unsubstituted alkenyl groups have from 2 to 8 carbon atoms.

The phrase “substituted alkenyl” has the same meaning with respect to unsubstituted alkenyl groups that substituted alkyl groups had with respect to unsubstituted alkyl groups. A substituted alkenyl group includes alkenyl groups in which a non-carbon or non-hydrogen atom is bonded to a carbon double bonded to another carbon and those in which one of the non-carbon or non-hydrogen atoms is bonded to a carbon not involved in a double bond to another carbon.

The phrase “unsubstituted alkynyl” refers to straight and branched chain groups such as those described with respect to unsubstituted alkyl groups as defined above, except that at least one triple bond exists between two carbon atoms. Examples include, but are not limited to —C≡C(H), —C≡C(CH₃), —C≡C(CH₂CH₃), —C(H₂)C≡C(H), —C(H)₂C≡C(CH₃), and —C(H)₂C≡C(CH₂CH₃) among others. In some embodiments, unsubstituted alkynyl groups have from 2 to 8 carbon atoms.

The phrase “substituted alkynyl” has the same meaning with respect to unsubstituted alkynyl groups that substituted alkyl groups had with respect to unsubstituted alkyl groups. A substituted alkynyl group includes alkynyl groups in which a non-carbon or non-hydrogen atom is bonded to a carbon triple bonded to another carbon and those in which a non-carbon or non-hydrogen atom is bonded to a carbon not involved in a triple bond to another carbon.

The phrase “unsubstituted heterocyclyl” refers to both aromatic and nonaromatic ring compounds including monocyclic, bicyclic, and polycyclic ring compounds such as, but not limited to, quinuclidyl, containing 3 or more ring members of which one or more is a heteroatom such as, but not limited to, N, O, and S. Although the phrase “unsubstituted heterocyclyl” includes condensed heterocyclic rings such as benzimidazolyl, it does not include heterocyclyl groups that have other groups such as alkyl or halo groups bonded to one of the ring members as compounds such as 2-methylbenzimidazolyl are substituted heterocyclyl groups. Examples of heterocyclyl groups include, but are not limited to: unsaturated 3 to 8 membered rings containing 1 to 4 nitrogen atoms such as, but not limited to pyrrolyl, pyrrolinyl, imidazolyl, pyrazolyl, pyridinyl, dihydropyridinyl, pyrimidyl, pyrazinyl, pyridazinyl, triazolyl (e.g. 4H-1,2,4-triazolyl, 1H-1,2,3-triazolyl, 2H-1,2,3-triazolyl etc.), tetrazolyl, (e.g. 1H-tetrazolyl, 2H tetrazolyl, etc.); saturated 3 to 8 membered rings containing 1 to 4 nitrogen atoms such as, but not limited to, pyrrolidinyl, imidazolidinyl, piperidinyl, piperazinyl; condensed unsaturated heterocyclic groups containing 1 to 4 nitrogen atoms such as, but not limited to, indolyl, isoindolyl, indolinyl, indolizinyl, benzimidazolyl, quinolyl, isoquinolyl, indazolyl, benzotriazolyl; unsaturated 3 to 8 membered rings containing 1 to 2 oxygen atoms and 1 to 3 nitrogen atoms such as, but not limited to, oxazolyl, isoxazolyl, oxadiazolyl (e.g. 1,2,4-oxadiazolyl, 1,3,4-oxadiazolyl, 1,2,5-oxadiazolyl, etc.); saturated 3 to 8 membered rings containing 1 to 2 oxygen atoms and 1 to 3 nitrogen atoms such as, but not limited to, morpholinyl; unsaturated condensed heterocyclic groups containing 1 to 2 oxygen atoms and 1 to 3 nitrogen atoms, for example, benzoxazolyl, benzoxadiazolyl, benzoxazinyl (e.g. 2H-1,4-benzoxazinyl etc.); unsaturated 3 to 8 membered rings containing 1 to 3 sulfur atoms and 1 to 3 nitrogen atoms such as, but not limited to, thiazolyl, isothiazolyl, thiadiazolyl (e.g. 1,2,3-thiadiazolyl, 1,2,4-thiadiazolyl, 1,3,4-thiadiazolyl, 1,2,5-thiadiazolyl, etc.); saturated 3 to 8 membered rings containing 1 to 2 sulfur atoms and 1 to 3 nitrogen atoms such as, but not limited to, thiazolodinyl; saturated and unsaturated 3 to 8 membered rings containing 1 to 2 sulfur atoms such as, but not limited to, thienyl, dihydrodithiinyl, dihydrodithionyl, tetrahydrothiophene, tetrahydrothiopyran; unsaturated condensed heterocyclic rings containing 1 to 2 sulfur atoms and 1 to 3 nitrogen atoms such as, but not limited to, benzothiazolyl, benzothiadiazolyl, benzothiazinyl (e.g. 2H-1,4-benzothiazinyl, etc.), dihydrobenzothiazinyl (e.g. 2H-3,4-dihydrobenzothiazinyl, etc.), unsaturated 3 to 8 membered rings containing oxygen atoms such as, but not limited to furyl; unsaturated condensed heterocyclic rings containing 1 to 2 oxygen atoms such as benzodioxolyl (e.g. 1,3-benzodioxoyl, etc.); unsaturated 3 to 8 membered rings containing an oxygen atom and 1 to 2 sulfur atoms such as, but not limited to, dihydrooxathiinyl; saturated 3 to 8 membered rings containing 1 to 2 oxygen atoms and 1 to 2 sulfur atoms such as 1,4-oxathiane; unsaturated condensed rings containing 1 to 2 sulfur atoms such as benzothienyl, benzodithiinyl; and unsaturated condensed heterocyclic rings containing an oxygen atom and 1 to 2 oxygen atoms such as benzoxathiinyl. Heterocyclyl group also include those described above in which one or more S atoms in the ring is double-bonded to one or two oxygen atoms (sulfoxides and sulfones). For example, heterocyclyl groups include tetrahydrothiophene oxide, and tetrahydrothiophene 1,1-dioxide. Preferred heterocyclyl groups contain 5 or 6 ring members. More preferred heterocyclyl groups include morpholine, piperazine, piperidine, pyrrolidine, imidazole, pyrazole, 1,2,3-triazole, 1,2,4-triazole, tetrazole, thiophene, thiomorpholine, thiomorpholine in which the S atom of the thiomorpholine is bonded to one or more O atoms, pyrrole, homopiperazine, oxazolidin-2-one, pyrrolidin-2-one, oxazole, quinuclidine, thiazole, isoxazole, furan, and tetrahydrofuran.

The phrase “substituted heterocyclyl” refers to an unsubstituted heterocyclyl group as defined above in which one or more of the ring members is bonded to a non-hydrogen atom such as described above with respect to substituted alkyl groups and substituted aryl groups. Examples, include, but are not limited to, 2-methylbenzimidazolyl, 5-methylbenzimidazolyl, 5-chlorobenzthiazolyl, N-alkyl piperazinyl groups such as 1-methyl piperazinyl, piperazine-N-oxide, N-alkyl piperazine N-oxides, 2-phenoxy-thiophene, and 2-chloropyridinyl among others. In addition, substituted heterocyclyl groups also include heterocyclyl groups in which the bond to the non-hydrogen atom is a bond to a carbon atom that is part of a substituted and unsubstituted aryl, substituted and unsubstituted aralkyl, or unsubstituted heterocyclyl group. Examples include but are not limited to 1-benzylpiperidinyl, 3-phenythiomorpholinyl, 3-(pyrrolidin-1-yl)-pyrrolidinyl, and 4-(piperidin-1-yl)-piperidinyl. Groups such as N-alkyl substituted piperazine groups such as N-methyl piperazine, substituted morpholine groups, and piperazine N-oxide groups such as piperazine N-oxide and N-alkyl piperazine N-oxides are examples of some substituted heterocyclyl groups. Groups such as substituted piperazine groups such as N-alkyl substituted piperazine groups such as N-methyl piperazine and the like, substituted morpholine groups, piperazine N-oxide groups, and N-alkyl piperazine N-oxide groups are examples of some substituted heterocyclyl groups that are especially suited as R⁶ or R⁷ groups.

The phrase “unsubstituted heterocyclylalkyl” refers to unsubstituted alkyl groups as defined above in which a hydrogen or carbon bond of the unsubstituted alkyl group is replaced with a bond to a heterocyclyl group as defined above. For example, methyl (—CH₃) is an unsubstituted alkyl group. If a hydrogen atom of the methyl group is replaced by a bond to a heterocyclyl group, such as if the carbon of the methyl were bonded to carbon 2 of pyridine (one of the carbons bonded to the N of the pyridine) or carbons 3 or 4 of the pyridine, then the compound is an unsubstituted heterocyclylalkyl group.

The phrase “substituted heterocyclylalkyl” has the same meaning with respect to unsubstituted heterocyclylalkyl groups that substituted aralkyl groups had with respect to unsubstituted aralkyl groups. However, a substituted heterocyclylalkyl group also includes groups in which a non-hydrogen atom is bonded to a heteroatom in the heterocyclyl group of the heterocyclylalkyl group such as, but not limited to, a nitrogen atom in the piperidine ring of a piperidinylalkyl group. In addition, a substituted heterocyclylalkyl group also includes groups in which a carbon bond or a hydrogen bond of the alkyl part of the group is replaced by a bond to a substituted and unsubstituted aryl or substituted and unsubstituted aralkyl group. Examples include but are not limited to phenyl-(piperidin-1-yl)-methyl and phenyl-(morpholin-4-yl)-methyl.

The phrase “substituted heterocyclyloxy” refers to a hydroxyl group (—OH) in which the bond to the hydrogen atom is replaced by a bond to a ring atom of an otherwise substituted heterocyclyl group as defined above.

The phrase “unsubstituted aryloxyalkyl” refers to an unsubstituted alkyl group as defined above in which a carbon bond or hydrogen bond is replaced by a bond to an oxygen atom which is bonded to an unsubstituted aryl group as defined above.

The phrase “substituted aryloxyalkyl” refers to an unsubstituted aryloxyalkyl group as defined above in which a bond to a carbon or hydrogen group of the alkyl group of the aryloxyalkyl group is bonded to a non-carbon and non-hydrogen atom as described above with respect to substituted alkyl groups or in which the aryl group of the aryloxyalkyl group is a substituted aryl group as defined above.

The phrase “unsubstituted heterocyclyloxyalkyl” refers to an unsubstituted alkyl group as defined above in which a carbon bond or hydrogen bond is replaced by a bond to an oxygen atom which is bonded to an unsubstituted heterocyclyl group as defined above.

The phrase “substituted heterocyclyloxyalkyl” refers to an unsubstituted heterocyclyloxyalkyl group as defined above in which a bond to a carbon or hydrogen group of the alkyl group of the heterocyclyloxyalkyl group is bonded to a non-carbon and non-hydrogen atom as described above with respect to substituted alkyl groups or in which the heterocyclyl group of the heterocyclyloxyalkyl group is a substituted heterocyclyl group as defined above.

The phrase “unsubstituted heterocyclylalkoxy” refers to an unsubstituted alkyl group as defined above in which a carbon bond or hydrogen bond is replaced by a bond to an oxygen atom which is bonded to the parent compound, and in which another carbon or hydrogen bond of the unsubstituted alkyl group is bonded to an unsubstituted heterocyclyl group as defined above.

The phrase “substituted heterocyclylalkoxy” refers to an unsubstituted heterocyclylalkoxy group as defined above in which a bond to a carbon or hydrogen group of the alkyl group of the heterocyclylalkoxy group is bonded to a non-carbon and non-hydrogen atom as described above with respect to substituted alkyl groups or in which the heterocyclyl group of the heterocyclylalkoxy group is a substituted heterocyclyl group as defined above. Further, a substituted heterocyclylalkoxy group also includes groups in which a carbon bond or a hydrogen bond to the alkyl moiety of the group may be substituted with one or more additional substituted and unsubstituted heterocycles. Examples include but are not limited to pyrid-2-ylmorpholin-4-ylmethyl and 2-pyrid-3-yl-2-morpholin-4-ylethyl.

The phrase “unsubstituted alkoxyalkyl” refers to an unsubstituted alkyl group as defined above in which a carbon bond or hydrogen bond is replaced by a bond to an oxygen atom which is bonded to an unsubstituted alkyl group as defined above.

The phrase “substituted alkoxyalkyl” refers to an unsubstituted alkoxyalkyl group as defined above in which a bond to a carbon or hydrogen group of the alkyl group and/or the alkoxy group of the alkoxyalkyl group is bonded to a non-carbon and non-hydrogen atom as described above with respect to substituted alkyl groups.

The term “protected” with respect to hydroxyl groups, amine groups, and sulfhydryl groups refers to forms of these functionalities which are protected from undesirable reaction with a protecting group known to those skilled in the art such as those set forth in Protective Groups in Organic Synthesis, Greene, T. W.; Wuts, P. G. M., John Wiley & Sons, New York, N.Y., (3rd Edition, 1999) which can be added or removed using the procedures set forth therein. Examples of protected hydroxyl groups include, but are not limited to, silyl ethers such as those obtained by reaction of a hydroxyl group with a reagent such as, but not limited to, t-butyldimethyl-chlorosilane, trimethylchlorosilane, triisopropylchlorosilane, triethylchlorosilane; substituted methyl and ethyl ethers such as, but not limited to methoxymethyl ether, methythiomethyl ether, benzyloxymethyl ether, t-butoxymethyl ether, 2-methoxyethoxymethyl ether, tetrahydropyranyl ethers, 1-ethoxyethyl ether, allyl ether, benzyl ether; esters such as, but not limited to, benzoylformate, formate, acetate, trichloroacetate, and trifluoracetate. Examples of protected amine groups include, but are not limited to, amides such as, formamide, acetamide, trifluoroacetamide, and benzamide; imides, such as phthalimide, and dithiosuccinimide; and others. Examples of protected sulfhydryl groups include, but are not limited to, thioethers such as S-benzyl thioether, and S-4-picolyl thioether; substituted S-methyl derivatives such as hemithio, dithio and aminothio acetals; and others.

Pharmaceutical combinations of the present invention include (a) at least one RTK inhibitor compound selected from the group consisting of compounds of Formula I or a tautomer thereof, compounds of Formula II or a tautomer thereof, compounds of Formula III or a tautomer thereof, a pharmaceutically acceptable salt of the compound, a pharmaceutically acceptable salt of the tautomer, or a mixture thereof.

The RTK inhibitor compound may be selected from a compound of formula I, a tautomer of the compound, a salt of the compound, a salt of the tautomer, or a mixture thereof, wherein the compound of formula I has the following formula:

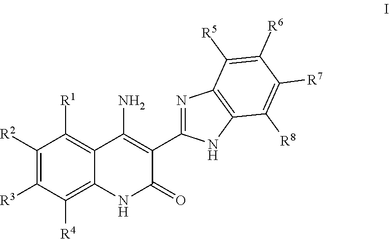

wherein:

R¹, R², R³, and R⁴ may be the same or different and are independently selected from H, Cl, Br, F, I, —OR¹⁰ groups, —NR¹¹R¹² groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, or substituted or unsubstituted heterocyclylalkyl groups;

R⁵, R⁶, R⁷, and R^{S} may be the same or different and are independently selected from H, Cl, Br, F, I, —OR¹³ groups, —NR¹⁴R¹⁵ groups, —SR¹¹ groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, substituted or unsubstituted heterocyclylalkyl groups, substituted or unsubstituted alkoxyalkyl groups,

substituted or unsubstituted aryloxyalkyl groups, or substituted or unsubstituted heterocyclyloxyalkyl groups;

R¹⁰ and R¹³ may be the same or different and are independently selected from substituted or unsubstituted alkyl groups, substituted or unsubstituted aryl groups, substituted or unsubstituted heterocyclyl groups, substituted or unsubstituted heterocyclylalkyl groups, substituted or unsubstituted alkoxyalkyl groups, substituted or unsubstituted aryloxyalkyl groups, or substituted or unsubstituted heterocyclyloxyalkyl groups;

R¹¹ and R¹⁴ may be the same or different and are independently selected from substituted or unsubstituted alkyl groups, substituted or unsubstituted aryl groups, or substituted or unsubstituted heterocyclyl groups;

R¹² and R¹⁵ may be the same or different and are independently selected from substituted or unsubstituted alkyl groups, substituted or unsubstituted aryl groups, or substituted or unsubstituted heterocyclyl groups; and

R¹⁶ is selected from substituted or unsubstituted alkyl groups, substituted or unsubstituted aryl groups, or substituted or unsubstituted heterocyclyl groups.

The RTK inhibitor compound may also be selected from a compound of Formula II or a tautomer thereof, a pharmaceutically acceptable salt of the compound, a pharmaceutically acceptable salt of the tautomer, or a mixture thereof, wherein the compound of formula II has the following formula:

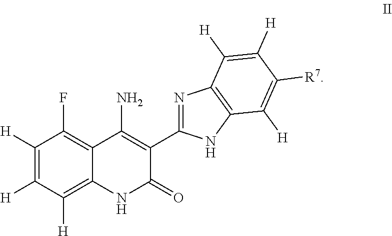

wherein:

R⁷ is a substituted or unsubstituted heterocyclyl group. In some embodiments, R⁷ is a substituted or unsubstituted heterocyclyl group selected from a substituted or unsubstituted piperidinyl group, piperazinyl group, or morpholinyl group. In other embodiments, R⁷ is a substituted or unsubstituted N-alkyl piperazinyl group. In further embodiments, R⁷ is a substituted or unsubstituted N-alkyl piperazinyl group and the alkyl group of the N-alkyl piperazinyl comprises from 1 to 4 carbon atoms.

The RTK inhibitor compound may also be selected from a compound of Formula III or a tautomer thereof, a pharmaceutically acceptable salt of the compound, a pharmaceutically acceptable salt of the tautomer, or a mixture thereof, wherein the compound of formula III has the following formula:

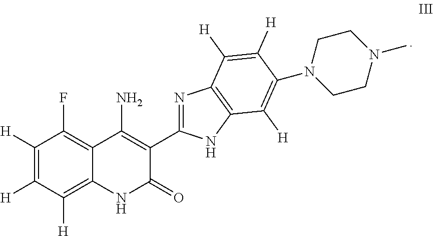 [SMILES: CN1CCN(c2ccc3nc(-c4c(N)c5c(F)cccc5[nH]c4=O)[nH]c3c2)CC1]

Compounds of Formula III include 4-amino-5-fluoro-3-[5-(4-methylpiperazin-1-yl)-1H-benzimidazol-2-yl]quinolin-2(1H)-one (Compound A) and (4-amino-5-fluoro-3-[6-(4-methylpiperazin-1-yl)-1H-benzimidazol-2-yl]quinolin-2(1H)-one) (Compound B).

In a preferred embodiment, the pharmaceutical combination of the present invention includes at least one compound of Formula I or a tautomer thereof, compound of Formula II or a tautomer thereof, compound of Formula III or a tautomer thereof, a pharmaceutically acceptable salt of the compound, a pharmaceutically acceptable salt of the tautomer, or a mixture thereof that is Compound A.

In another preferred embodiment, the pharmaceutical combination of the present invention includes at least one compound of Formula I or a tautomer thereof, compound of Formula II or a tautomer thereof, compound of Formula III or a tautomer thereof, a pharmaceutically acceptable salt of the compound, a pharmaceutically acceptable salt of the tautomer, or a mixture thereof that is Compound B.

The RTK inhibitor compounds of Formula I or a tautomer thereof, compounds of Formula II or a tautomer thereof, compounds of Formula III or a tautomer thereof, a pharmaceutically acceptable salt of the compound, a pharmaceutically acceptable salt of the tautomer, or a mixture thereof; formulations of same, and methods for preparing same are described in, for example, WO2002/222598, WO2003/087095, WO2005/046589, WO2006/127926, WO2006/124413, WO2007/064719, WO2009/115562 and WO2012/001074 which are hereby incorporated by reference in entirety.

The compound of the invention may be administered in free form or in pharmaceutically acceptable salt form.

A “pharmaceutically acceptable salt”, as used herein, unless otherwise indicated, includes a salt with an inorganic base, organic base, inorganic acid, organic acid, or basic or acidic amino acid. As salts of inorganic bases, the invention includes, for example, alkali metals such as sodium or potassium; alkaline earth metals such as calcium and magnesium or aluminum; and ammonia. As salts of organic bases, the invention includes, for example, trimethylamine, triethylamine, pyridine, picoline, ethanolamine, diethanolamine, and triethanolamine. As salts of inorganic acids, the instant invention includes, for example, hydrochloric acid, hydroboric acid, nitric acid, sulfuric acid, and phosphoric acid. As salts of organic acids, the instant invention includes, for example, formic acid, acetic acid, trifluoroacetic acid, fumaric acid, oxalic acid, tartaric acid, maleic acid, lactic acid, citric acid, succinic acid, malic acid, methanesulfonic acid, benzenesulfonic acid, and p-toluenesulfonic acid. As salts of basic amino acids, the instant invention includes, for example, arginine, lysine and ornithine. Acidic amino acids include, for example, aspartic acid and glutamic acid.

The monolactate salt of the compound of Formula I exists in a variety of polymorphs, including, e.g., the monohydrate form and the anhydrous form. Polymorphs occur where the same composition of matter (including its hydrates and solvates) crystallizes in a different lattice arrangement resulting in different thermodynamic and physical properties specific to the particular crystalline form.

Additional pharmaceutically acceptable salts of Compound A and Compound B suitable for the present invention include the salts disclosed in WO2005/04658, which is hereby incorporated into the present application by reference.

Pharmaceutical combinations of the present invention further include (b) at least one anti-estrogen compound, or a pharmaceutically acceptable salt thereof. Anti-estrogen compounds are e.g., compounds which antagonize the effect of estrogens at the estrogen receptor level. The term includes, but is not limited to, tamoxifen, toremifene, fulvestrant, raloxifene and raloxifene hydrochloride.

Tamoxifen, or tamoxifen citrate, also known as NOLVADEX, is a nonsteroidal estrogen antagonist. Tamoxifen citrate, also known as (Z)2-[4-(1,2-diphenyl-1-butenyl)phenoxy]-N,N-dimethylethanamine 2 hydroxy-1,2,3-propanetricarboxylate (1:1), is a trans-isomer of a triphenylethylene derivative having the following structure:

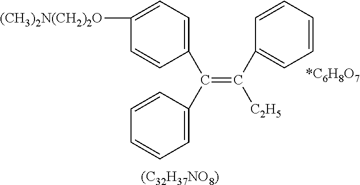

Tamoxifen, also known as (Z)-2-[4-(1,2-Diphenyl-1-butenyl)phenoxy]-N,N-dimethyl ethanamine, or 1-p-β-dimethylamino ethoxyphenyl-trans-1,2-diphenylbut-1-ene, has the following structure:

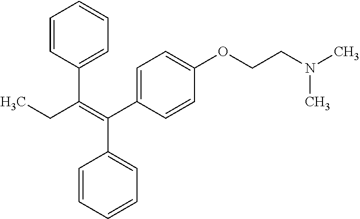 [SMILES: CC/C(=C(\c1ccccc1)c1ccc(OCCN(C)C)cc1)c1ccccc1]

Tamoxifen and methods for its preparation have been described, e.g. in U.S. Pat. No. 4,536,516.

Toremifene or toremifene citrate, also known as FARESTON, is a nonsteroidal estrogen agonist/antagonist. Toremifene, also known as 2-[4-[(1Z)-4-Chloro-1,2-diphenyl-1-buten-1-yl]phenoxy]-N,N-dimethylethanamine or (Z)-4-chloro-1,2-diphenyl-1-[4-[2-(N,N-dimethylamino)ethoxy]phenyl]-1-butene, has the following structure:

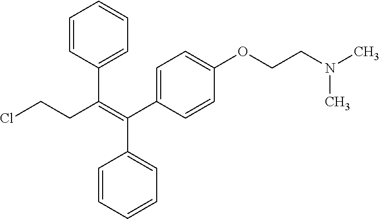 [SMILES: CN(C)CCOc1ccc(/C(=C(/CCCl)c2ccccc2)c2ccccc2)cc1]

Toremifene and methods for its preparation have been described, e.g. in U.S. Pat. No. 4,696,949.

Fulvestrant, also known as FASLODEX, is a steroidal estrogen receptor antagonist reported to lack any partial agonist activity. Fulvestrant, also known as (7α,17β)-7-[9-[(4,4,5,5,5-Pentafluoropentyl)sulfinyl]nonyl]estra-1,3,5(10)-triene-3,17-diol, has the following structure:

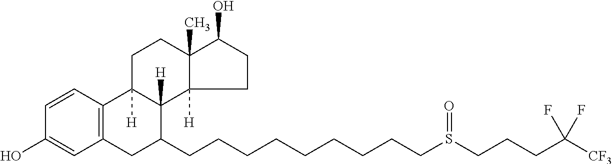

Fulvestrant and methods for its preparation have been described, e.g. in U.S. Pat. No. 4,659,516.

Raloxifene, or raloxifene hydrochloride, also known as EVISTA, is a nonsteroidal estrogen agonist/antagonist, commonly referred to as a selective estrogen receptor modulator (SERM). Raloxifene, also known as keoxifene or [6-hydroxy-2-(4-hydroxyphenyl)benzo[b]thien-3-yl]-[4-[2-(1-piperidinyl)ethoxy]phenyl]methanone, is a benzothiophene having the following structure:

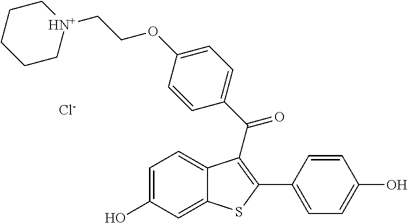 [SMILES: O=C(c1ccc(OCC[NH+]2CCCCC2)cc1)c1c(-c2ccc(O)cc2)sc2cc(O)ccc12 ; [Cl-]]

Raloxifene and methods for its preparation have been described, e.g. in U.S. Pat. No. 4,418,068.

In a preferred embodiment, the pharmaceutical combination of the present invention includes at least one anti-estrogen compound that is tamoxifen.

In another preferred embodiment, the pharmaceutical combination of the present invention includes at least one anti-estrogen compound that is toremifene.

In another preferred embodiment, the pharmaceutical combination of the present invention includes at least one anti-estrogen compound that is fulvestrant.

In a further preferred embodiment, the pharmaceutical combination of the present invention includes at least one anti-estrogen compound that is raloxifene.

Unless otherwise specified, or clearly indicated by the text, reference to therapeutic agents useful in the pharmaceutical combination of the present invention includes both the free base of the compounds, and all pharmaceutically acceptable salts of the compounds.

The structure of the compounds identified by code nos., generic or trade names may be taken from the actual edition of the standard compendium “The Merck Index” or from databases, e.g., Patents International (IMS World Publications). The corresponding content thereof is hereby incorporated by reference.

In each case where citations of patent applications are given herein, the subject matter relating to the compounds is hereby incorporated into the present application by reference. The compounds used as therapeutic agents in the pharmaceutical combinations of the present invention can be prepared and administered as described in the cited documents, respectively. Comprised are likewise the pharmaceutically acceptable salts thereof, the corresponding racemates, diastereoisomers, enantiomers, tautomers, as well as the corresponding crystal modifications of above disclosed compounds where present, e.g. solvates, hydrates and polymorphs, which are disclosed therein. Also within the scope of this invention is the combination of two separate therapeutic agents as set forth above, i.e., a pharmaceutical combination within the scope of this invention could include three therapeutic agents or more.

A pharmaceutical combination which comprises (a) at least one RTK inhibitor compound selected from the group consisting of compounds of Formula I or a tautomer thereof, compounds of Formula II or a tautomer thereof, compounds of Formula III or a tautomer thereof, a pharmaceutically acceptable salt of the compound, a pharmaceutically acceptable salt of the tautomer, or a mixture thereof; and (b) one or more anti-estrogen compounds, or a pharmaceutically acceptable salt thereof; such as tamoxifen, toremifene, fulvestrant, raloxifene or raloxifene hydrochloride, will be referred to hereinafter as a COMBINATION OF THE INVENTION.

According to the present invention, the preferred combination partners are (a) a RTK inhibitor compound selected from the group consisting of 4-amino-5-fluoro-3-[5-(4-methylpiperazin-1-yl)-1H-benzimidazol-2-yl]quinolin-2(1H)-one (Compound A) and (4-amino-5-fluoro-3-[6-(4-methylpiperazin-1-yl)-1H-benzimidazol-2-yl]quinolin-2(1H)-one) (Compound B) or a pharmaceutically acceptable salt thereof, and (b) a least one anti-estrogen which is tamoxifen, toremifene, fulvestrant, raloxifene or a pharmaceutically acceptable salt thereof.

In another embodiment, the preferred combination partners are (a) at least one RTK inhibitor compound which is 4-amino-5-fluoro-3-[5-(4-methylpiperazin-1-yl)-1H-benzimidazol-2-yl]quinolin-2(1H)-one (Compound A) or a pharmaceutically acceptable salt thereof, and (b) at least one anti-estrogen which is fulvestrant or a pharmaceutically acceptable salt thereof.

The present invention also pertains to a combination such as a combined preparation or a pharmaceutical composition which comprises (a) a RTK inhibitor compound selected from the group consisting of 4-amino-5-fluoro-3-[5-(4-methylpiperazin-1-yl)-1H-benzimidazol-2-yl]quinolin-2(1H)-one (Compound A) and (4-amino-5-fluoro-3-[6-(4-methylpiperazin-1-yl)-1H-benzimidazol-2-yl]quinolin-2(1H)-one) (Compound B) or a pharmaceutically acceptable salt thereof, and (b) a least one anti-estrogen which is tamoxifen, toremifene, fulvestrant, raloxifene or a pharmaceutically acceptable salt thereof.

The present invention particularly pertains to a COMBINATION OF THE INVENTION useful for treating or preventing a proliferative disease in a subject in need thereof. In this embodiment of the present invention, the COMBINATION OF THE INVENTION is used for the treatment or prevention of a proliferative disease comprising administering to the subject a combination therapy, comprising an effective amount of a RTK inhibitor compound targeting/decreasing a protein or lipid kinase activity selected from COMPOUND A or COMPOUND B and an effective amount of an anti-estrogen compound. Preferably, these compounds are administered at therapeutically effective dosages which, when combined, provide a beneficial effect. The administration may be simultaneous or sequential.

In one embodiment, the proliferative disease is cancer. The term “cancer” is used herein to mean a broad spectrum of tumors, including all solid tumors and hematological malignancies. Examples of such tumors include but are not limited to benign or malignant tumors of the brain and central nervous system, lung (in particular small-cell lung cancer and non-small cell lung cancer), bladder, gastric, pancreatic, breast, head and neck, renal, kidney, ureter, ovarian, prostate, colorectal, esophageal, testicular, gynecological (e.g., ovarian, uterine sarcomas, carcinoma of the fallopian tubes, endometrial, cervix, vagina or vulva), thyroid, pancreatic, bone, skin, melanoma, rectal, anal, colon, testicular, Hodgkin's disease, small intestine, endocrine system (e.g., thyroid, parathyroid, or adrenal glands), soft tissue and bone sarcoma, urethra, penis, leukemia, lymphomas, multiple myeloma, biliary, liver, neurofibromatosis, acute myelogenous leukemia (AML), myelodysplastic syndromes (MDS), and Kaposi's sarcoma.

In a further embodiment of the present invention, the proliferative disease is melanoma, lung cancer (including non-small cell lung cancer (NSCLC)), colorectal cancer (CRC), breast cancer, kidney cancer such as e.g., renal cell carcinoma (RCC), liver cancer, endometrial cancer, acute myelogenous leukemia (AML), myelodysplastic syndromes (MDS), thyroid cancer, pancreatic cancer, neurofibromatosis or hepatocellular carcinoma.

In a further embodiment of the present invention, the proliferative disease is a solid tumor. The term “solid tumor” especially means breast cancer, ovarian cancer, colorectal cancer, and generally gastrointestinal tract, cervix cancer, lung cancer (including small-cell lung cancer and non-small cell lung cancer), head and neck cancer, bladder cancer, prostate cancer or Kaposi's sarcoma. The present combination inhibits the growth of solid tumors and also hematological tumors. Further, depending on the tumor type and particular combination used, a decrease of the tumor volume can be obtained. The COMBINATION OF THE INVENTION disclosed herein is also suited to prevent the metastatic spread of tumors and the growth or development of micrometastases. The COMBINATION OF THE INVENTION disclosed herein are suitable for the treatment of poor prognosis patients, especially such poor prognosis patients having breast cancer.

In a further embodiment, the proliferative disease is human breast cancers (e.g. primary breast tumours, invasive ductal or lobular adenocarcinomas of the breast); and endometrial cancers.

In a further embodiment, the proliferative disease is breast cancer, particularly hormone receptor positive (HR+) breast cancer or HR+/HER2− breast cancer.

In a further embodiment, the breast cancer to be treated is HR+/HER2− breast cancer where patients have evidence of disease progression on prior endocrine therapy.

It will be understood that the COMBINATION OF THE INVENTION may be used solely for the treatment of a proliferative disease in accordance with the present invention.

The COMBINATION OF THE INVENTION is particularly useful for the treatment of cancers having amplification of the fibroblast growth factor (FGF) pathway, particularly cancers having amplification of the FGFR1 gene, which is associated with resistance to endocrine therapy. In one embodiment, the cancer to be treated is HR+/HER2− breast cancer where patients have amplification of the FGFR1 gene. In a further embodiment, the cancer to be treated is HR+/HER2− breast cancer where patients have amplification of the FGFR1 gene and where patients have evidence of disease progression on prior endocrine therapy.

It has been found that the combination therapy comprising a RTK inhibitor compound 4-amino-5-fluoro-3-[5-(4-methylpiperazin-1-yl)-1H-benzimidazol-2-yl]quinolin-2(1H)-one (Compound A) or (4-amino-5-fluoro-3-[6-(4-methylpiperazin-1-yl)-1H-benzimidazol-2-yl]quinolin-2(1H)-one) (Compound B) with an anti-estrogen, particularly fulvestrant, results in significant improvement in the treatment or prevention of proliferative diseases as compared to the monotherapy. When administered simultaneously, sequentially or separately, the RTK inhibitor compound 4-amino-5-fluoro-3-[5-(4-methylpiperazin-1-yl)-1H-benzimidazol-2-yl]quinolin-2(1H)-one (Compound A) or (4-amino-5-fluoro-3-[6-(4-methylpiperazin-1-yl)-1H-benzimidazol-2-yl]quinolin-2(1H)-one) (Compound B) and the anti-estrogen interact significantly to inhibit cell proliferation. The COMBINATIONS OF THE INVENTION are in particular suitable for the treatment of patients with advanced cancer who have failed standard systemic therapy. This includes patients having tumor types showing resistance to monotherapy or showing resistance to combinations different from those disclosed herein.

The nature of proliferative diseases is multifactorial. Under certain circumstances, drugs with different mechanisms of action may be combined. However, just considering any combination of therapeutic agents having different mode of action does not necessarily lead to combinations with advantageous effects.

The administration of a pharmaceutical combination of the invention results not only in a beneficial effect, e.g. a significant therapeutic effect, e.g. with regard to alleviating, delaying progression of or inhibiting the symptoms, but also in further significant beneficial effects, e.g. fewer side-effects, an improved quality of life or a decreased morbidity.

It can be shown by established test models that a COMBINATION OF THE INVENTION results in the beneficial effects described herein before. The person skilled in the art is fully enabled to select a relevant test model to prove such beneficial effects. The pharmacological activity of a COMBINATION OF THE INVENTION may, for example, be demonstrated in a clinical study or in a test procedure as essentially described hereinafter.

Suitable clinical studies are in particular, for example, open label, dose escalation studies in patients with a proliferative diseases. Such studies prove in particular the synergism of the therapeutic agents of the COMBINATION OF THE INVENTION. The beneficial effects on proliferative diseases may be determined directly through the results of these studies which are known as such to a person skilled in the art. Such studies may be, in particular, be suitable to compare the effects of a monotherapy using either therapeutic agent and a COMBINATION OF THE INVENTION. In one embodiment, the dose of a RTK inhibitor compound selected from the group consisting of COMPOUND A or COMPOUND B is escalated until the Maximum Tolerated Dosage is reached, and at least one anti-estrogen is administered with a fixed dose. Alternatively, a RTK inhibitor compound selected from the group consisting of COMPOUND A or COMPOUND B may be administered in a fixed dose and the dose of at least one anti-estrogen inhibitor may be escalated. Each patient may receive doses of a RTK inhibitor compound selected from the group consisting of COMPOUND A or COMPOUND B either daily or intermittently. The efficacy of the treatment is determined in such studies, e.g., after 12, 18 or 24 weeks by evaluation of symptom scores every 6 weeks, or by evaluating the delay in progression and tumor reduction.

Determining a synergistic interaction between one or more components, the optimum range for the effect and absolute dose ranges of each component for the effect may be definitively measured by administration of the components over different w/w ratio ranges and doses to patients in need of treatment. For humans, the complexity and cost of carrying out clinical studies on patients may render impractical the use of this form of testing as a primary model for synergy. However, the observation of synergy in one species can be predictive of the effect in other species and animal models exist, as described herein, to measure a synergistic effect and the results of such studies can also be used to predict effective dose and plasma concentration ratio ranges and the absolute doses and plasma concentrations required in other species by the application of pharmacokinetic pharmacodynamic methods. Established correlations between tumor models and effects seen in man suggest that synergy in animals may, e.g., be demonstrated in breast cancer HCT-116 cells.

In a preferred embodiment of the present invention, the COMBINATION OF THE INVENTION comprises the RTK inhibitor COMPOUND A and at least one anti-estrogen that is fulvestrant for use in the treatment or prevention of a proliferative disease, preferably a cancer, comprising an amplification of the FGF pathway. Preferably, the amplification of the FGF pathway is an amplification of FGFR1. Preferably, the cancer comprising an amplification of the FGF pathway or FGFR1 is breast. Preferably, the breast cancer is HR+ breast cancer, or HR+/HER2− breast cancer. More preferably, the cancer to be treated is HR+/HER2− breast cancer where patients have evidence of disease progression on prior endocrine therapy.

In one aspect, the present invention provides a synergistic combination for human administration comprising (a) a RTK inhibitor compound selected from the group consisting of 4-amino-5-fluoro-3-[5-(4-methylpiperazin-1-yl)-1H-benzimidazol-2-yl]quinolin-2(1H)-one (Compound A) and (4-amino-5-fluoro-3-[6-(4-methylpiperazin-1-yl)-1H-benzimidazol-2-yl]quinolin-2(1H)-one) (Compound B) or a pharmaceutically acceptable salt thereof, and (b) a least one anti-estrogen, particularly fulvestrant, or a pharmaceutically acceptable salt thereof, in a combination range (w/w) which corresponds to the ranges observed in a tumor model, e.g., as described in the Examples below, used to identify a synergistic interaction. Suitably, the ration range in humans corresponds to a non-human range selected from between 50:1 to 1:50 parts by weight, 50:1 to 1:20,50:1 to 1:10,50:1 to 1:1,20:1 to 1:50,20:1 to 1:20,20:1 to 1:10,20:1 to 1:1, 10:1 to 150, 10:1 to 1:20, 10:1 to 1:10, 10:1 to 1:1, 1:1 to 1:50, 1:1 to 1:20 and 1:1 to 1:10. More suitably, the human range corresponds to a non-human range of the order of 10:1 to 1:1, 5:1 to 1:1 or 2:1 to 1:1 parts by weight.

According to a further aspect, the present invention provides a synergistic combination for administration to humans comprising (a) a RTK inhibitor compound selected from the group consisting of 4-amino-5-fluoro-3-[5-(4-methylpiperazin-1-yl)-1H-benzimidazol-2-yl]quinolin-2(1H)-one (Compound A) and (4-amino-5-fluoro-3-[6-(4-methylpiperazin-1-yl)-1H-benzimidazol-2-yl]quinolin-2(1H)-one) (Compound B) or a pharmaceutically acceptable salt thereof, and (b) a least one anti-estrogen, particularly fulvestrant, or a pharmaceutically acceptable salt thereof, where the dose range of each component corresponds to the synergistic ranges observed in a suitable tumor model, e.g., the tumor models described in the Examples below, primarily used to identify a synergistic interaction.

It is one objective of this invention to provide a pharmaceutical composition comprising a quantity, which is jointly therapeutically effective against a proliferative disease comprising the COMBINATION OF THE INVENTION. In this composition, the combination partners (a) and (b) can be either administered in a single formulation or unit dosage form, administered concurrently but separately, or administered sequentially by any suitable route. The unit dosage form may also be a fixed combination.

The pharmaceutical compositions for separate administration of both combination partners, or for the administration in a fixed combination, i.e. a single galenical composition comprising the COMBINATION OF THE INVENTION, may be prepared in a manner known per se and are those suitable for enteral, such as oral or rectal, and parenteral administration to mammals (warm-blooded animals), including humans, comprising a therapeutically effective amount of at least one pharmacologically active combination partner alone, e.g. as indicated above, or in combination with one or more pharmaceutically acceptable carriers, especially suitable for enteral or parenteral application.

The novel pharmaceutical composition contains may contain, from about 0.1% to about 99.9%, preferably from about 1% to about 60%, of the therapeutic agent(s).

Suitable pharmaceutical compositions for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of various conventional mixing, comminution, direct compression, granulating, sugar-coating, dissolving, lyophilizing processes, or fabrication techniques readily apparent to those skilled in the art. It will be appreciated that the unit content of a combination partner contained in an individual dose of each dosage form need not in itself constitute an effective amount since the necessary effective amount may be reached by administration of a plurality of dosage units.

A unit dosage form containing the combination of agents or individual agents of the combination of agents may be in the form of micro-tablets enclosed inside a capsule, e.g. a gelatin capsule. For this, a gelatin capsule as is employed in pharmaceutical formulations can be used, such as the hard gelatin capsule known as CAPSUGEL, available from Pfizer. The unit dosage forms of the present invention may optionally further comprise additional conventional carriers or excipients, used for pharmaceuticals. Examples of such carriers include, but are not limited to, disintegrants, binders, lubricants, glidants, stabilizers, and fillers, diluents, colorants, flavours and preservatives. One of ordinary skill in the art may select one or more of the aforementioned carriers with respect to the particular desired properties of the dosage form by routine experimentation and without any undue burden. The amount of each carriers used may vary within ranges conventional in the art. The following references which are all hereby incorporated by reference disclose techniques and excipients used to formulate oral dosage forms. See The Handbook of Pharmaceutical Excipients, 4^{th} edition, Rowe et al., Eds., American Pharmaceuticals Association (2003); and Remington: the Science and Practice of Pharmacy, 2oth edition, Gennaro, Ed., Lippincott Williams & Wilkins (2003).

These optional additional conventional carriers may be incorporated into the oral dosage form either by incorporating the one or more conventional carriers into the initial mixture before or during granulation or by combining the one or more conventional carriers with granules comprising the combination of agents or individual agents of the combination of agents in the oral dosage form. In the latter embodiment, the combined mixture may be further blended, e.g., through a V-blender, and subsequently compressed or molded into a tablet, for example a monolithic tablet, encapsulated by a capsule, or filled into a sachet.

Examples of pharmaceutically acceptable disintegrants include, but are not limited to, starches; clays; celluloses; alginates; gums; cross-linked polymers, e.g., cross-linked polyvinyl pyrrolidone; cross-linked sodium carboxymethylcellulose or croscarmellose sodium, e.g., ACDI-SOL from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 5% by weight of composition.

Examples of pharmaceutically acceptable binders include, but are not limited to, starches; celluloses and derivatives thereof, for example, microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder may be present in an amount from about 0% to about 50%, e.g., 2-20% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight. Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 0% to about 80% by weight of the composition.

In one embodiment, the present invention also pertains to a COMBINATION OF THE INVENTION for use in the preparation of a pharmaceutical composition or medicament for the treatment or prevention of a proliferative disease in a subject in need thereof.

In a further embodiment, the present invention pertains to the use of a RTK inhibitor compound selected from the group consisting of 4-amino-5-fluoro-3-[5-(4-methylpiperazin-1-yl)-1H-benzimidazol-2-yl]quinolin-2(1H)-one (Compound A) and (4-amino-5-fluoro-3-[6-(4-methylpiperazin-1-yl)-1H-benzimidazol-2-yl]quinolin-2(1H)-one) (Compound B) or a pharmaceutically acceptable salt thereof, in combination with at least one anti-estrogen, particularly fulvestrant, or a pharmaceutically acceptable salt thereof, for the preparation of a pharmaceutical composition or medicament for the treatment or prevention of a proliferative disease in a subject in need thereof.

In accordance with the present invention, a therapeutically effective amount of each of the combination partner of the COMBINATION OF THE INVENTION may be administered simultaneously or sequentially and in any order, and the components may be administered separately or as a fixed combination. For example, the method of treating a proliferative disease according to the invention may comprise (i) administration of the first agent (a) in free or pharmaceutically acceptable salt form and (ii) administration of an agent (b) in free or pharmaceutically acceptable salt form, simultaneously or sequentially in any order, in jointly therapeutically effective amounts, preferably in synergistically effective amounts, e.g. in daily or intermittently dosages corresponding to the amounts described herein. The individual combination partners of the COMBINATION OF THE INVENTION may be administered separately at different times during the course of therapy or concurrently in divided or single combination forms. Furthermore, the term “administering” also encompasses the use of a prodrug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the COMBINATION OF THE INVENTION may vary depending on the particular compound or pharmaceutical composition employed, the mode of administration, the condition being treated, and the severity of the condition being treated. Thus, the dosage regimen of the COMBINATION OF THE INVENTION is selected in accordance with a variety of factors including the route of administration and the renal and hepatic function of the patient. A clinician or physician of ordinary skill can readily determine and prescribe the effective amount of the single therapeutic agents required to alleviate, counter or arrest the progress of the condition.

The optimum ratios, individual and combined dosages, and concentrations of the combination partners (a) and (b) of the COMBINATION OF THE INVENTION that yield efficacy without toxicity are based on the kinetics of the therapeutic agents' availability to target sites, and are determined using methods known to those of skill in the art.

The effective dosage of each of the combination partners may require more frequent administration of one of the compound(s) as compared to the other compound(s) in the combination. Therefore, to permit appropriate dosing, packaged pharmaceutical products may contain one or more dosage forms that contain the combination of compounds, and one or more dosage forms that contain one of the combination of compounds, but not the other compound(s) of the combination.

When the combination partners, which are employed in the COMBINATION OF THE INVENTION, are applied in the form as marketed as single drugs, their dosage and mode of administration can be in accordance with the information provided on the package insert of the respective marketed drug, if not mentioned herein otherwise.

The RTK inhibitor COMPOUND A may be administered to a suitable subject daily in single or divided doses at an effective dosage in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. For example, the RTK inhibitor COMPOUND A may also be administered to a suitable subject in a single dose of 500 mg per day, for 5 days on/2 days off dosing schedule (i.e. patients may take COMPOUND A on Day 1 through Day 5, and may take no medication on Day 6 and Day 7 “rest days”).

The RTK inhibitor COMPOUND B may be administered daily to a suitable subject in single or divided doses at an effective dosage in the range of about 0.001 to about 100 mg per kg body weight per day, preferably about 1 mg/kg/day to about 35 mg/kg/day, in single or divided doses. For a 70 kg human, this would amount to about 0.07 to 2.45 g/day, preferably about 0.05 to about 1.0 g/day.

The anti-estrogen may be administered to a suitable subject in single or divided doses at an effective dosage in the range of about 0.001 to 1000 mg and more preferred from 1.0 to 30 mg/kg body weight daily. Dosage unit compositions may contain such amounts of submultiples thereof to make up the daily dose. A total daily dose administered to a host in single or divided doses may be in amounts, for example, of from 0.001 to 1000 mg/kg body weight daily and from 1.0 to 30 mg/kg body weight daily. Dosage unit compositions may contain such amounts of submultiples thereof to make up the daily dose.

Fulvestrant may be administered to a suitable subject in a single dose administered on days 1, 15, 29 and once monthly thereafter at an effective dose in the range of about 250 or 500 mg.

Toremifene may be administered to a suitable subject in a single daily dose of 60 mg.

Tamoxifen may be administered to a suitable subject in single daily dose of 20 mg.

Raloxifene may be administered to a suitable subject in a single daily dose of 60 mg.

The optimal dosage of each combination partner for treatment of a proliferative disease can be determined empirically for each individual using known methods and will depend upon a variety of factors, including, though not limited to, the degree of advancement of the disease; the age, body weight, general health, gender and diet of the individual; the time and route of administration; and other medications the individual is taking. Optimal dosages may be established using routine testing and procedures that are well known in the art.

The amount of each combination partner that may be combined with the carrier materials to produce a single dosage form will vary depending upon the individual treated and the particular mode of administration. In some embodiments the unit dosage forms containing the combination of agents as described herein will contain the amounts of each agent of the combination that are typically administered when the agents are administered alone.

Frequency of dosage may vary depending on the compound used and the particular condition to be treated or prevented. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

The present invention relates to a method of treating a subject having a proliferative disease comprising administered to said subject a COMBINATION OF THE INVENTION in a quantity, which is jointly therapeutically effective against a proliferative disease. In particular, the proliferative disease to be treated with a COMBINATION OF THE INVENTION is a breast cancer, particularly HR+ breast cancer or HR+/HER2− breast cancer, or HR+/HER2− breast cancer where patients have evidence of disease progression on prior endocrine therapy. Furthermore, the treatment can comprise surgery or radiotherapy.

The present invention further relates to the COMBINATION OF THE INVENTION for use in the treatment of a proliferative disease, particularly cancer.

The present invention further provides a commercial package comprising as therapeutic agents COMBINATION OF THE INVENTION, together with instructions for simultaneous, separate or sequential administration thereof for use in the delay of progression or treatment of a proliferative disease in a subject in need thereof.

The following Examples illustrate the invention described above; they are not, however, intended to limit the scope of the invention in any way. The beneficial effects of the pharmaceutical combination of the present invention can also be determined by other test models known as such to the person skilled in the pertinent art.

### Example 1

### HR+/HER2− Breast Cancer

Postmenopausal HER2−/HR+ locally advanced or metastatic breast cancer patients (about 150 total patients) progressing within 12 months of completion of adjuvant endocrine therapy or after ≦1 prior endocrine therapy in the advanced setting, i.e. that have evidence of disease progression on or after prior endocrine therapy and the cancer is not amenable to curative treatment by surgery or radiotherapy, are enrolled in a multicenter, randomized, double blind, placebo controlled, phase II trial. Patients undergo molecular screening to enrich for FGF-amplification (FGFR1 and/or FGFR2 and/or FGF3 amplification by qPCR; 45 patients per arm). Specific inclusion/exclusion criteria include:

Inclusion Criteria:
1. Postmenopausal women with HER2−, HR+ locally advanced or metastatic breast cancer
2. Progression on or after endocrine treatment
3. Measureable disease as per RECIST or evaluable bone disease
4. ECOG 0, 1 or 2

Exclusion Criteria:
1. Evidence of CNS or leptomenigeal metastases
2. Previous treatment with fulvestrant
3. Previous chemotherapy for locally advanced or metastatic breast cancer
4. Cirrhosis or chronic active/persistent hepatitis

Patients are randomized 1:1 to receive fulvestrant in combination with oral Compound A or placebo until disease progression, unacceptable toxicity, or death. Eligible patients receive open label fulvestrant in combination with the study drug Compound A. Open-Label Fulvestrant (in solution) is injected intramuscularly at a dose of 500 mg once on Week 1 Day 1, Week 3 Day 1 and Week 5 Day 1 and subsequently once every 4 weeks on Day 1 of the week. In addition, active Compound A (in tablet form) is taken orally at a dose of 500 mg (i.e., 5×100 mg tablets) on a 5 days on/2 days off dosing schedule (i.e. patients will take COMPOUND A on Day 1 through Day 5, and will take no medication on Day 6 and Day 7 “rest days”).

The primary endpoint is progression-free survival (PFS), with tumor assessments performed every 8 weeks. PFS is defined as the date of randomization to the date of the first radiologically documented disease progression (PD) or death due to any cause per local investigator assessment as per Response Evaluation Criteria in Solid Tumors (RECIST v. 1.1). Additional endpoints include overall response rate per RECIST v1.1, duration of response, overall survival, ECOG performance status and patient reported outcome scores over time, and safety:
(a) Overall Response Rate (ORR), is measured every 8 weeks. ORR is defined as the percentage of patients with a best overall response of Complete Response (CR) or Partial Response (PR) as per RECIST v. 1.1;
(b) Duration of Response (DOR), is measured from the date of first documented efficacy response (CR or PR) to time of documented progression (PD). DOR is defined as time from the date of the first documented response (CR or PR) to the date of the first documented PD or death due to disease. If a patient does not have a progression event, DOR is censored on the date of the last adequate tumor assessment;
(c) Overall Survival (OS), is measured from the date of randomization to the date of death from any cause. OS is defined as the time from the date of randomization to the date of death from any cause. If a patient is not known to have died at the date of analysis cut-off, the OS is censored at the last date of contact;
(d) Safety (type, frequency and severity of adverse events, and laboratory values), is measured at screening, Week 2, Week 4 and approximately every 4 weeks during treatment period. The type, frequency and severity of adverse events, laboratory values, and Electrocardiograms (ECGs) experienced by patients are assessed according to Common Terminology Criteria for Adverse Events; and
(e) Eastern Cooperative Oncology Group (ECOG) Performance Status (scales and criteria used by doctors and researchers to assess how a patient's disease is progressing and assess how the disease affects the daily living abilities of the patient.) is measured at screening, every 4 weeks during treatment period, and every 8 weeks during follow-up. The time to worsening of ECOG performance status is measured.
   Compound A in combination with fulvestrant is superior to fulvestrant plus placebo in either the FGF amplified subpopulation or in the full population.

The pharmacodynamic effect of Compound A on FGFR-associated angiogenic pathways in tumor specimens is explored. Specifically, the baseline levels of a number of tumor markers is measured in archival tumor biopsies (FGFR receptor and bFGF levels) and the change from baseline of FGF pathway markers in paired biopsies obtained during the study (incl. bFGF, pFGFR and pFRS2).

Multiple predictive biomarkers of response to Compound A signalling modulation are assessed through the collection of and analysis of archival and/or fresh tumor biopsies, and the most suitable markers for assessing the effect of Compound A at molecular level and on clinical outcome, in particular:
(a) pFGFR, pFRS2, and pERK (IHC) are markers that measure the pharmacodynamic effects of Compound A;
(b) cleaved caspase-3,Ki-67 are markers that reflect anti-tumor effects of Compound A at a molecular or cellular level; and
(c) FGFR1, FGFR2, and FGF3 amplification are markers that predict patient response to FGFR inhibitors.

Markers (a) and (b) are selected for measuring pharmacodynamic effect and are used to understand how the target inhibition correlates with downstream molecular effects and cellular responses in this patient population. Markers (c) are selected as potential markers predicting patient response to the treatment regimen. Signalling through TK receptors (RTKs) is critical for cell proliferation and survival. Compound A inhibits cellular proliferation and/or induces apoptosis which then reflects in increased anti-tumor effects of the therapy.

### Example 2

### ER+/FGFR1 Amplified BREAST CANCER Cell Lines

CAMA-1 cell line was obtained from ATCC, catalogue #HTB-21. Cells were cultured in either EMEM (ATCC #30-2003) supplemented with 10% FBS (Gibco, Catalogue #10099-141), or EMEM without phenol red (Invitrogen, catalogue #51200) and supplemented with 10% FBS that had been treated with charcoal-dextran to remove steroids (Invitrogen catalogue #12676-029). Cell lines were grown at 37° C. and 5% CO₂. To split and expand, cells were dislodged from flasks using 0.25% Trypsin-EDTA (Corning catalogue#25-053-CI). Since phenol red has a structural similarity to some estrogens, cells were also dislodged from flask using 0.25% TrypLE Express without phenol red (Invitrogen catalogue #12604-013).

Cells were dispensed into tissue culture treated 96-well plates (Costar, catalogue#3904) in a final volume of 80 ul of medium at a density of 4000 cells per well. 24 hours following plating, 20 ul of each compound dilution series was transferred to plates containing the cells, resulting in compound concentration ranges from 50 nM to 4000 nM by 3-fold dilutions and a final DMSO concentration of 0.16%. The total volume in each well was 100 ul and each dose matrix was tested in triplicate on separate assay plates. Plates were incubated for 72 hrs. and the effects of the compounds on cell proliferation was determined using the CellTiter-Glo™ Luminescent Cell Viability Assay (CTG,Promega catalogue#7573) and a Victor™ X4 plate reader (Perkin Elmer).

Compound B was ordered as powder from the Novartis archive, dissolved in 100% DMSO (Cellgro, catalogue#25-290-CQC) at a concentration of 10 mM and stored at −20° C. until use. Fulvestrant was obtained from Sigma-Aldrich as a powder (catalogue #14409) and dissolved in 100% ethanol at a concentration of 10 mM and stored at 20° C. until use. Compounds were arrayed in a 3 ml deep 12-well reservoir and serially diluted 3-fold six times yielding concentration ranges from 300 nM to 24000 nM.

Data analysis of cell proliferation was performed using Chalice Analyzer as described in Lehar et al. 2009. The average and standard deviation percent inhibition relative to DMSO treated control for triplicate compound-treated wells at each concentration of compounds was determined and up-loaded to Chalice Analyzer Database.

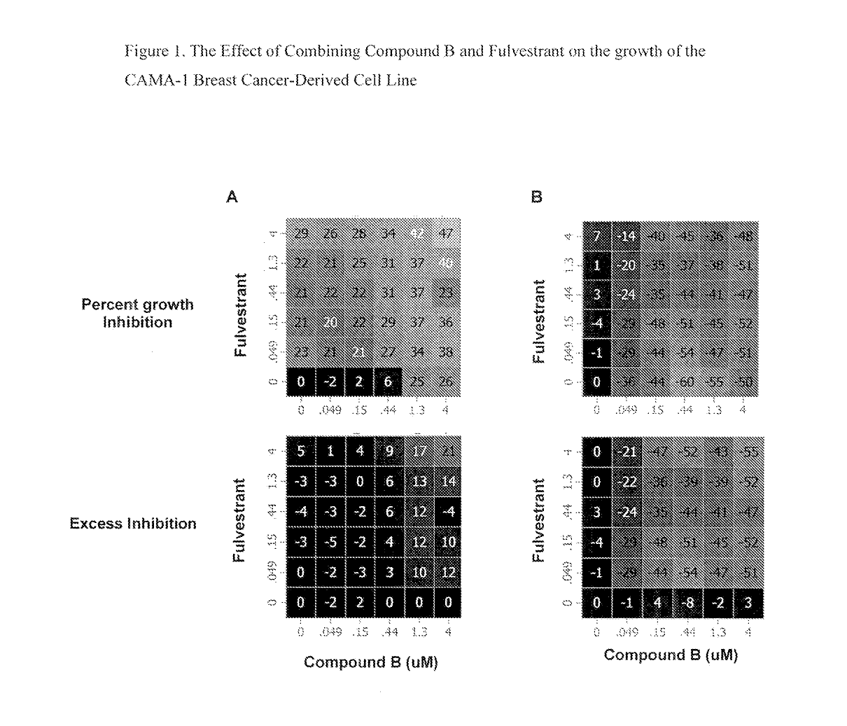

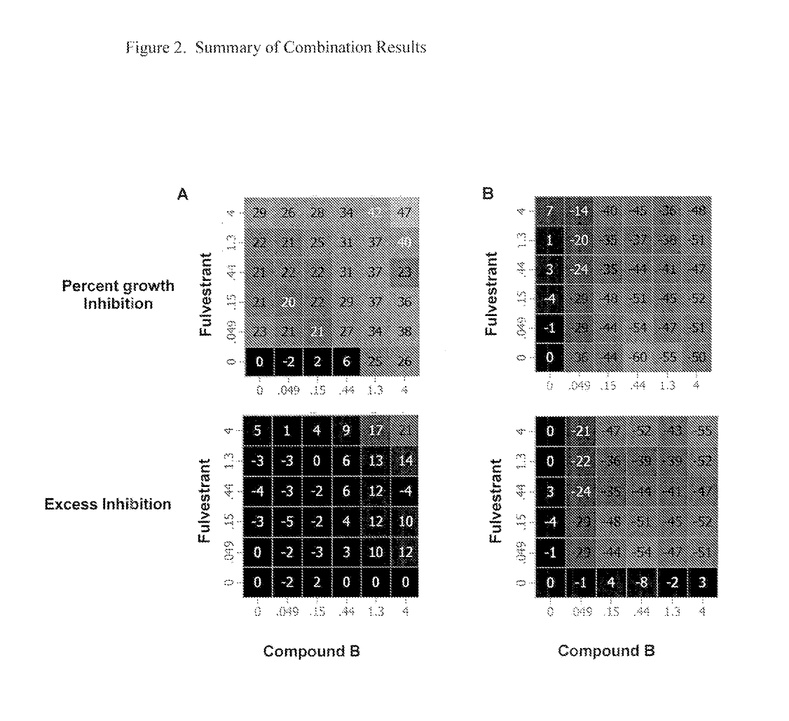

No combination effects were observed between the compounds in steroid-depleted media (synergy score=0), however Compound B and fulvestrant resulted in synergy as shown in FIGS. 1A and 2A in standard media containing steroid, suggesting that combination effects observed in A are steroid dependent.

1. A pharmaceutical combination comprising:
(a) a RTK inhibitor compound comprising a compound of formula I, a tautomer of the compound, a salt of the compound, a salt of the tautomer, or a mixture thereof, wherein the compound of formula I has the following formula: 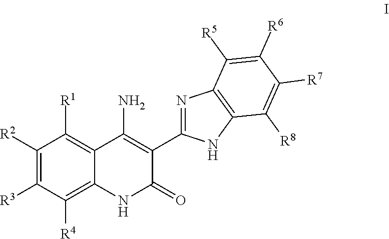
wherein:
R¹, R², R³, and R⁴ may be the same or different and are independently selected from H, Cl, Br, F, I, —OR¹⁰ groups, —NR¹¹R¹² groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, or substituted or unsubstituted heterocyclylalkyl groups:
R⁵, R⁶, R⁷, and R⁸ may be the same or different and are independently selected from H, Cl, Br, F, I, —OR¹³ groups, —NR¹⁴R¹⁵ groups, —SR¹¹ groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, substituted or unsubstituted heterocyclylalkyl groups, substituted or unsubstituted alkoxyalkyl groups, substituted or unsubstituted aryloxyalkyl groups, or substituted or unsubstituted heterocyclyloxyalkyl groups;
R¹⁰ and R¹³ may be the same or different and are independently selected from substituted or unsubstituted alkyl groups, substituted or unsubstituted aryl groups, substituted or unsubstituted heterocyclyl groups, substituted or unsubstituted heterocyclylalkyl groups, substituted or unsubstituted alkoxyalkyl groups, substituted or unsubstituted aryloxyalkyl groups, or substituted or unsubstituted heterocyclyloxyalkyl groups;
R¹¹ and R¹⁴ may be the same or different and are independently selected from substituted or unsubstituted alkyl groups, substituted or unsubstituted aryl groups, or substituted or unsubstituted heterocyclyl groups;
R¹² and R¹⁵ may be the same or different and are independently selected from substituted or unsubstituted alkyl groups, substituted or unsubstituted aryl groups, or substituted or unsubstituted heterocyclyl groups; and
R¹⁶ is selected from substituted or unsubstituted alkyl groups, substituted or unsubstituted aryl groups, or substituted or unsubstituted heterocyclyl groups: and
(b) at least one anti-estrogen or a pharmaceutically acceptable salt thereof, for simultaneous, separate or sequential administration.

2. A pharmaceutical combination according to claim 1, wherein the RTK inhibitor is a compound of Formula II or a tautomer thereof, a pharmaceutically acceptable salt of the compound, a pharmaceutically acceptable salt of the tautomer, or a mixture thereof, wherein the compound of formula II has the following formula and R⁷ is a substituted or unsubstituted heterocyclyl group:

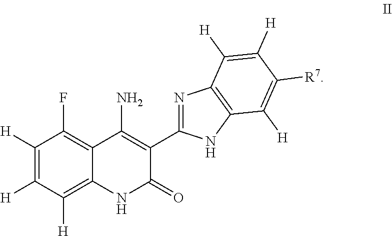

3. A pharmaceutical combination according to claim 2, wherein R⁷ is a substituted or unsubstituted heterocyclyl group selected from a substituted or unsubstituted piperidinyl group. piperazinyl group, or morpholinyl group.

4. A pharmaceutical combination according to claim 3, wherein R⁷ is a substituted or unsubstituted N-alkyl piperazinyl group.

5. A pharmaceutical combination according to claim 4, wherein R⁷ is a substituted or unsubstituted N-alkyl piperazinyl group and the alkyl group of the N-alkyl piperazinyl comprises from 1 to 4 carbon atoms.

6. A pharmaceutical combination according to claim 1, wherein the RTK inhibitor is a compound of Formula III or a tautomer thereof, a pharmaceutically acceptable salt of the compound, a pharmaceutically acceptable salt of the tautomer, or a mixture thereof, wherein the compound of formula III has the following formula:

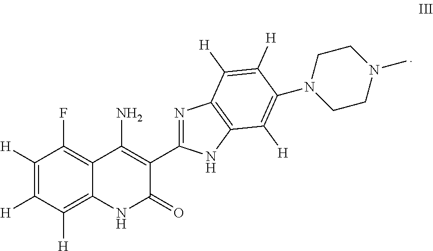 [SMILES: CN1CCN(c2ccc3nc(-c4c(N)c5c(F)cccc5[nH]c4=O)[nH]c3c2)CC1]

7. A pharmaceutical combination according to claim 1, wherein the RTK inhibitor is 4-amino-5-fluoro-3-[5-(4-methylpiperazin-1-yl)-1H-benzimidazol-2-yl]quinolin-2(1H)-one.

8. A pharmaceutical combination according to claim 1, wherein the lactic acid salt of the compound is administered to the subject.

9. A pharmaceutical combination according to claim 1, wherein the anti-estrogen is tamoxifen, toremifene, fulvestrant, raloxifene, or a pharmaceutically acceptable salt thereof.

10. A pharmaceutical combination according to claim 1, wherein the anti-estrogen is fulvestrant.

11. A pharmaceutical combination according to claim 1 for use in the treatment of a proliferative disease in a subject in need thereof.

12. A pharmaceutical combination according to claim 1 for use in the preparation of a medicament for the treatment of a proliferative disease

13. A pharmaceutical combination according to claim 11, wherein the proliferative disease is cancer.

14. A pharmaceutical combination according to claim 12, wherein the proliferative disease is breast cancer, preferably HR+ or HER2− breast cancer, more preferably HR+/HER2− breast cancer.

15. A pharmaceutical combination according to claim 1, wherein the (a) a RTK inhibitor selected from the group consisting of 4-amino-5-fluoro-3-[5-(4-methylpiperazin-1-yl)-1H-benzimidazol-2-yl]quinolin-2(1H)-one or (4-amino-5-fluoro-3-[6-(4-methylpiperazin-1-yl)-1H-benzimidazol-2-yl]qunolin-2(1H)-one) or a pharmaceutically acceptable salt thereof, and (b) at least one anti-estrogen or a pharmaceutically acceptable salt thereof are provided in synergistically effective amounts for the treatment of a proliferative disease.

16. Use of the combination according to claim 1 for the manufacture of a medicament for the treatment of a proliferative disease.

17. Use according to claim 16, wherein the anti-estrogen is selected from tamoxifen, toremifene, fulvestrant, raloxifene and a pharmaceutically acceptable salt thereof.

18. Use according to claim 17, wherein the anti-estrogen is fulvestrant.

19. A method for treating a proliferative disease, comprising the simultaneous, separate or sequential administration of a therapeutically effective amount of a RTK inhibitor selected from 4-amino-5-fluoro-3-[5-(4-methylpiperazin-1-yl)-1H-benzimidazol-2-yl]quinolin-2(1H)-one or (4-amino-5-fluoro-3-[6-(4-methylpiperazin-1-yl)-1H-benzimidazol-2-yl]quinolin-2(1H)-one) or a pharmaceutically acceptable salt thereof, in combination with at least one anti-estrogen or a pharmaceutically acceptable salt thereof, to a patient having a proliferative disease.

20. A method according to claim 19, wherein the proliferative disease is breast cancer, preferably HR+ or HER2− breast cancer, more preferably HR+/HER2− breast cancer.

21. A combined preparation, which comprises (a) one or more unit dosage forms of a RTK inhibitor selected from the group consisting of 4-amino-5-fluoro-3-[5-(4-methylpiperazin-1-yl)-1H-benzimidazol-2-yl]quinolin-2(1H)-one or (4-amino-5-fluoro-3-[6-(4-methylpiperazin-1-yl)-1H-benzimidazol-2-yl]quinolin-2(1H)-one) or a pharmaceutically acceptable salt thereof, and (b) one or more unit dosage forms of at least one anti-estrogen or a pharmaceutically acceptable salt thereof.

22. A pharmaceutical composition comprising:
(a) a RTK inhibitor selected from the group consisting of 4-amino-5-fluoro-3-[5-(4-methylpiperazin-1-yl)-1H-benzimidazoil-2-yl]quinolin-2(1H)-one or (4-amino-5-fluoro-3-[6-(4-methylpiperazin-1-yl)-1H-benzimidazol-2-yl]quinolin-2(1H)-one) or a pharmaceutically acceptable salt thereof, and
(b) at least one anti-estrogen or a pharmaceutically acceptable salt thereof, for simultaneous, separate or sequential administration.

23. A pharmaceutical composition according to claim 22, wherein the RTK inhibitor and the anti-estrogen are provided in synergistically effective amounts for the treatment of a proliferative disease.

24. A pharmaceutical composition according to claim 23, wherein the proliferative disease is breast cancer, preferably HR+ or HER2− breast cancer, more preferably HR+/HER2− breast cancer.
